(19) United States
(12) Patent Application Publication
(10) Pub. No.: US 2013/0085348 A1
(43) Pub. Date: Apr. 4, 2013

(54) Method for Providing Remote Health Monitoring Data and Associated System

(71) Applicant: General Electric Company, Schenectady, NY (US)

(72) Inventors: Csaba DEVENYI, Budapest (HU); Roland LOHNER, Budapest (HU)

(73) Assignee: GENERAL ELECTRIC COMPANY, Schenectady, NY (US)

(21) Appl. No.: 13/631,108

(22) Filed: Sep. 28, 2012

(30) Foreign Application Priority Data
Sep. 30, 2011 (EP) 11462016.4

(51) Int. Cl. A61B 5/11; A61B 5/00

(52) U.S. Cl. 600/301; 600/300

(57) ABSTRACT

A method and a system for providing remote health monitoring data of an individual to be used in a health monitoring system is provided. The method comprising: measuring at least one vital sign of the individual; receiving and storing data representing the at least one vital sign obtained from the measurement; continuously measuring behavioral data of the individual; validating the data representing the at least one vital sign based on the behavioral data; and visually displaying information on a condition of the individual.

### BACKGROUND OF THE INVENTION

Embodiments of the invention relate generally to remote health monitoring, and in particular, to a method for providing remote health monitoring data in a reliable way. Embodiments of the invention further relate to a system for providing remote health monitoring data.

Health monitoring of individuals, especially of elderly people in their homes, is getting more and more important as hospitals are often overcrowded, too far or too expensive on a longer time basis. Many attempts have been made in the past in order to facilitate remote health care of elderly people, and to provide methods and systems for providing health monitoring data for care taking persons at different levels from medical specialists, through health care personnel to family members. On the basis of the collected information some kind of diagnosis can be repeatedly made by medical staff, medication instructions can be changed if necessary, or simply a signal may be given to family members when to call or visit an observed old-aged relative. In each case it is essential to collect reliable information which is not always possible with the currently used systems working in the homes of the individuals.

The patients are usually elderly people, usually having a chronic condition, and sometimes neurological diseases, which make their actions unpredictable. The measurements in most cases are performed by the patients, without any professional support, so it is hard to guarantee the proper execution of the measurement procedure.

There are many home monitoring systems available on the market. In most home monitoring systems available, it is possible for the patient to perform various measurements, and send the results to the physicians for review. The medical professionals supervising the monitoring of the patients make their decisions based on these measurement results. Thus the quality and reliability of this information is essential.

U.S. Pat. No. 7,684,999 discloses a user-based monitoring system including a remote user-based subsystem with at least one display and at least two microprocessor-based units in communication with each other. The subsystem is configured to facilitate collection of user-related data. The system also has at least one central server remotely located from, and configured for two-way communication with, the user-based subsystem so that it can receive and deliver signal communications to and from the user-based subsystem. The system also has at least one authorized user computer remotely located from, and configured for two-way signal communication with, the central server to receive user-related data collected by a remote user-based subsystem and allow an authorized user to communicate with the central server. The system is suited, amongst others, for monitoring remotely the health of a system user.

In the current commercial systems the measurements are performed by the patients, and it is up to the supervising medical professional to decide if the measured data is reliable.

Some systems, perform some validation of the data, but only based on the measurement result itself. For example, if the measured blood pressure was too high, the patient is asked if they took their proper medication, or if they performed some intensive activity before the measurement.

Due to the disadvantages of the prior art systems and methods, there is a continuous need for providing a method and system which makes it possible to improve reliability or to validate the results of measurements of elderly people or patients suffering from different diseases, carried out by the patients themselves.

### BRIEF DESCRIPTION OF THE INVENTION

In an embodiment, a method for providing remote health monitoring data of an individual to be used in a health monitoring system is provided. The method comprises measuring at least one vital sign of the individual, receiving and storing data representing the at least one vital sign obtained from the measurement, continuously measuring behavioral data of the individual, validating the data representing the at least one vital sign based on the behavioral data, and visually displaying information on a condition of the individual.

In another exemplary embodiment, a system for providing remote health monitoring data of an individual is provided. The system comprises a plurality of subsystems at a location of the individual, the plurality of subsystems comprising a subsystem control unit and at least one vital sign measuring unit configured to measure at least one vital sign of the individual, a central data server station in communication with the plurality of subsystems; and a monitoring side terminal in communication with the central station configured to provide information for visual display. At least a portion of the at least one vital sign measuring unit is in communication with the subsystem control unit. The subsystem control unit is connected to at least one behavioral data measuring unit configured to continuously measure behavioral data of the individual. The subsystem control unit is configured to receive and store the vital sign measurement and the continuous measurement of the behavioral data and to validate the vital sign measurement based on the continuous measurement of the behavioral data.

In another embodiment, a system for providing remote health monitoring data of an individual is provided. The system comprises a plurality of subsystems at a location of the individual, the plurality of subsystems comprising a subsystem control unit and at least one vital sign measuring unit configured to measure at least one vital sign of the individual, a central data server station in communication with the plurality of subsystems, and a monitoring side terminal in communication with the central station configured to provide information for visual display. At least a portion of the at least one vital sign measuring unit is in communication with the subsystem control unit. The subsystem control unit is connected with at least one behavioral data measuring unit configured to continuously measure behavioral data of the individual. The central data server unit is configured to receive and store the vital sign measurement and the continuous measurement of the behavioral data and to validate the vital sign measurement based on the continuous measurement of the behavioral data.

Further advantageous embodiments of the invention are provided in the depending claims.

### BRIEF DESCRIPTION OF THE DRAWINGS

Embodiments of the present invention will now be described in detail with reference to the accompanying drawing, in which:

FIG. 1 is a schematic bock diagram of an embodiment of a monitoring subsystem;

FIG. 2 is a schematic bock diagram of an embodiment of an extended system for performing remote monitoring;

FIG. 3 is a schematic diagram of an embodiment of a behavioral monitoring system in the home environment;

FIG. 4 is a schematic flow diagram of an embodiment of a method for performing vital sign measurements;

FIG. 5 is a schematic flow diagram of another an of a method for performing vital sign measurements;

FIG. 6 is a schematic flow diagram of an embodiment of a method for performing verified vital sign measurements; and

FIG. 7 is a schematic flow diagram of an embodiment of a method for performing verified vital sign measurements.

### DETAILED DESCRIPTION OF THE INVENTION

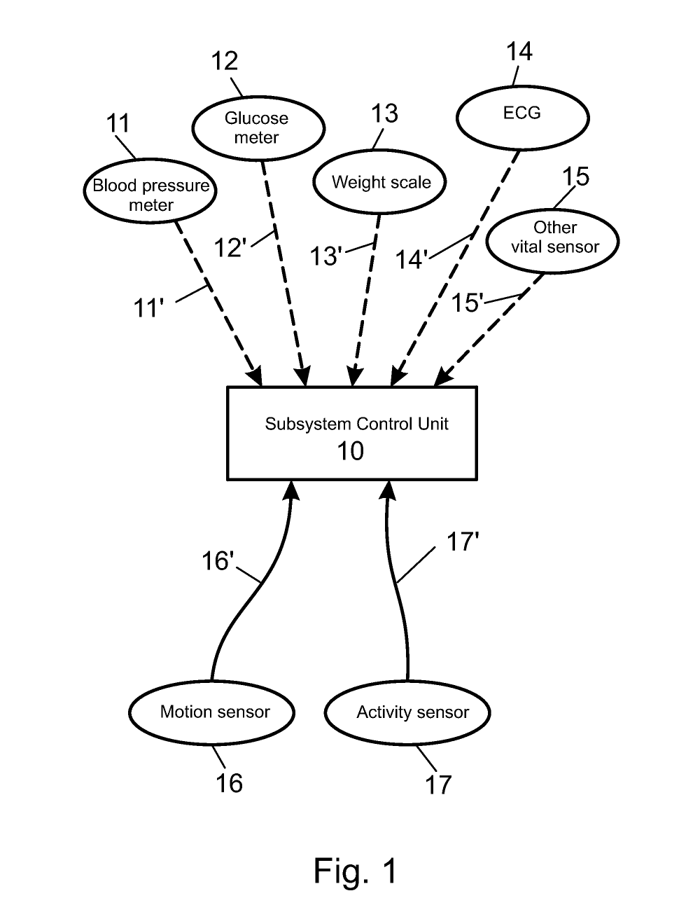

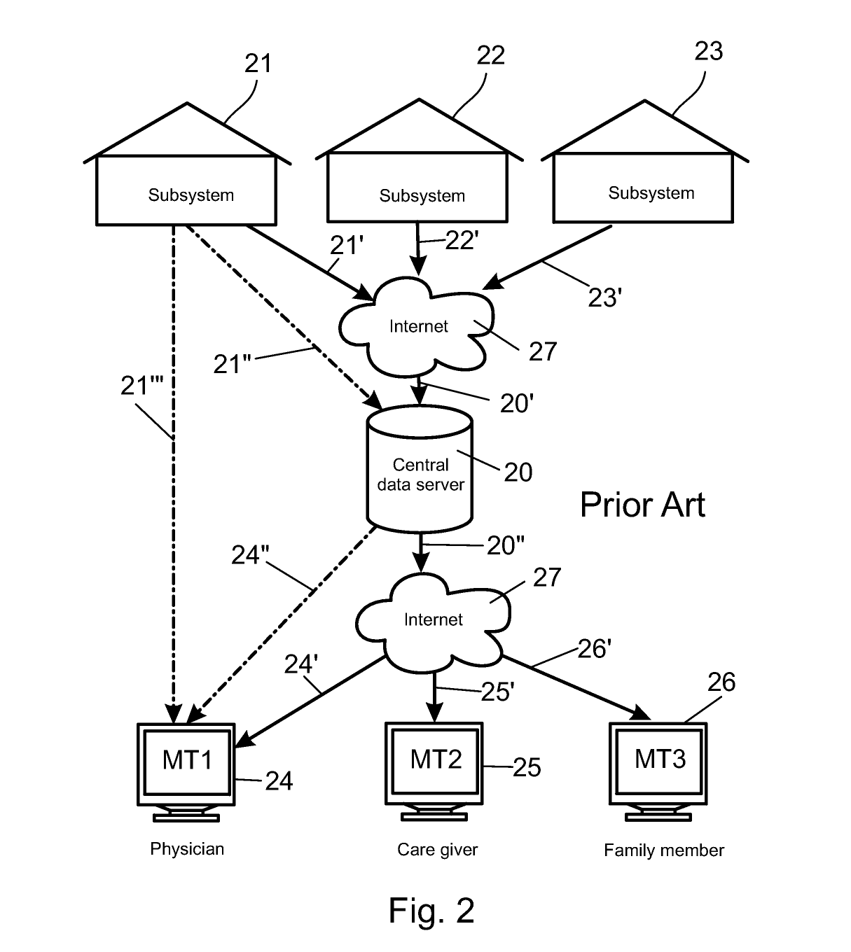

Referring first to FIG. 1, a schematic block diagram of an embodiment of a subsystem of a monitoring system is shown. The monitoring system will be explained in more detail on the basis of FIG. 2. The subsystem is provided with a subsystem control unit 10 (also called a home hub) and at least one measuring unit 11, 12, 13, 14, 15 for measuring vital signs of the individuals to be monitored. The measuring units 11, 12, 13, 14, 15 for measuring vital signs have a communication link to the subsystem control unit 10 as indicated by the dotted lines 11′, 12′, 13′, 14′, 15′. These communication links 11′, 12′, 13′, 14′, 15′ may be accomplished by wired communication links or by wireless communication links. The measuring units 11, 12, 13, 14, 15 for measuring vital signs of the individuals to be monitored may include for example, but not exclusively, a blood pressure meter 11 for measuring the blood pressure, a glucometer 12 for measuring the blood glucose, a weight scale 13 for determining the weight, an ECG monitor 14 for providing ECG data and other vital sign sensors 15 with the interaction of the monitored person. A portion or all of these measuring units 11, 12, 13, 14, 15 may have a wireless communication link to the subsystem control unit 10 as indicated by the dotted lines 11′, 12′, 13′, 14′, 15′. The wireless communication may be performed by using a radio communication according to Bluetooth, Zigbee, Wifi or other standardized or vendor specific proprietary specifications. Some of the devices that are outside the wireless communication range or do not have wireless communication capability, may be connected to the subsystem control unit 10 by a communication wire, such as a USB cable or the like. The vital sign measuring devices need not to be specialized devices. Any measurement device available off-the shelf may be suitable, even those which do not have any connection possibility. In this case, the home hub 10 used in the system must have a manual input capability to enable the user to input the measuring result obtained from such a measurement device, for example, a bathroom weight scale 13. When using such a device, the individual will read the result from the device and input (type) the value(s) using an input device (touchscreen, keyboard or similar) of the home hub. Such a subsystem for assisting elderly people or patients in carrying out vital sign measurement on their own, are known in the prior art such as is disclosed in U.S. Pat. No. 7,684,999.

An embodiment of the subsystem control unit 10 is also provided with at least one measuring unit 16, 17 for performing continuous measurement of behavioral data of the individuals to be monitored in addition to the distinct measurements of vital signs in order to increase reliability of the measurement of the vital signs without any substantial interaction of the monitored person. In this case, the subsystem control unit 10 is configured to receive and store the results of the distinct measurements of vital signs and of the continuous measurement of behavioral data. The subsystem control unit 10 uses the collected behavioral data to validate the data representing the vital signs of an individual to be monitored remotely. The measuring units 16, 17 for performing continuous measurement of behavioral data of the individuals to be monitored may include for example, but not exclusively, at least one fix mounted motion sensor 16 for determining the motion and/or location of the individual in a selected area, and/or at least one body-worn sensor 17 for sensing the activity such as speed and/or acceleration of the motion of a selected body part of the individual for determining the motion activity of the individual. The fix mounted motion sensors 16 may communicate with the subsystem control unit 10 using either a wireless or a wired communication link 16′. However, when using body-worn sensors 17, it may be more advantageous if the body-worn sensors 17 communicate with the subsystem control unit 10 using a wireless communication link 17′ so as not to restrict the wearer in his or her movement in any way. As a wired or wireless communication link 16′, 17′, the same or similar communication link may be selected as the ones used for the measuring unit 11, 12, 13, 14, 15 for measuring vital signs.

The fix mounted sensors 16 may be for example motion detectors or contact sensors mounted on walls or other pieces of furniture or equipments of the living area of the individuals to be monitored. These sensors 16 are not in direct contact with the monitored person. The contact sensors (not separately shown) can have different function in a monitored area. Typical installation points are the front door of the house/apartment, doors which might be useful to know if open or closed (e.g., bathroom door), doors of household equipment (e.g., door of the fridge) and places critical to healthcare related monitoring (e.g., if the person keeps all the medication in a closed drawer or box, the door of this holder). Motion sensors 16, such as passive infrared sensors (PIR sensors) NO can measure infrared light radiating from objects in its field of view. Actual motion is detected when an infrared source NO with one temperature changes its position in front of an infrared source with another temperature, for example, when a human passes the sensor's field of view in the monitored area. If there is a higher amount of motion then a pre-defined threshold, the sensor 16 sends a signal to the subsystem control unit 10.

The body-worn sensor 17 may be for example an activity sensor such as a speed or acceleration sensor fixed to a part of the body, preferably to a hand, arm, leg or foot of the wearer. These sensors 17 are in direct contact with the monitored person. The body-worn sensor or actigraph 17 as it is generally called, is body-worn equipment, most often worn on the wrist, like a wrist watch. The unit 17 continually records the movement of the equipment itself, therefore the movement of the patient's body part. This data can be used to calculate the motion of the monitored person (overall activity, step count, etc.).

The actigraph unit 17 generally consists of accelerometers to detect the acceleration of the unit along three axes, memory to store the recorded data until it is uploaded to a permanent storage, an interface, such as a Bluetooth unit, to communicate with the home hub and to send the collected data, and a battery

Also, a docking unit is part of the equipment. The docking unit has a power supply. The docking unit charges the battery of the actigraph when it is placed in the docking unit. Also, the actigraph 17 can detect the docking unit if the actigraph 17 is placed in the docking unit, and based on its settings, the actigraph 17 can initiate data transfer.

The actigraph 17 is normally worn by the monitored person during the hours of the day when the monitored person is awake, putting it on when he wakes up, and putting it into its docking unit before going to sleep at night. Therefore, during the hours of the day when the monitored person is awake, the actigraph 17 collects all motion performed by the monitored person, while during the hours of the day when the monitored person is asleep, the actigraph 17 charges the battery of the unit and transfers all collected data to a data storing unit, such as a home hub 10.

A remote health monitoring system using the above subsystem is schematically shown in FIG. 2. The system comprises a plurality of subsystems 21, 22, 23 at the location of the individuals to be monitored, the location being distant from medical assistance, such as in a home environment. In the simplest case, a subsystem, such as subsystem 21 may be connected to a monitoring terminal 24, 25, 26, such as monitoring terminal 24 via a communication channel 21′. The communication channel 21′ may be either a radio or a cable communication channel. In this case a monitoring person may have only access to one individual to be monitored at a time. Each change of the monitored person would require a reconnection to another communication channel. This problem can be solved by using a central communication and data server 20, which is capable of communicating with the subsystems 21, 22, 23 via data communication channels, through a cable or an air interface. According to one embodiment, the system also comprises a number of monitoring terminals 24, 25, 26 which are capable of communicating with the central server station 20 in order to provide information for visual display to the monitoring persons, such as health care professionals and/or care giving personnel and/or authorized family members.

Each group of the monitoring persons has a predetermined access right category to access monitoring information provided by the subsystems 21, 22, 23 and the central server unit 20. The health care professionals may, for example, be authorized to performing functionalities such as browsing patient data and setting up the monitoring parameters for the individual patient. The caregiver personnel may be authorized to browse patient data and prepare different reports based on it. The family members may be authorized to have access to their respective relative in order to have information about his or her health condition. The monitoring terminals 24, 25, 26 may be connected to the central server 20 through either a radio communication channel or a cable communication channel, or a combination of a radio communication channel and a cable communication channel 24′, 25′, 26′, such as the Internet 27. The use of the Internet as a communication channel makes it possible to set up the elements of the remote health monitoring system at any location of the world without limitation. Therefore in a most flexible configuration, the elements of the system, e.g., the subsystems 21, 22, 23 are connected through communication links 21′, 22′, 23′, the central server unit 20 through communication links 20′ and 20″, and the monitoring terminals 24, 25, 26 through communication links 24′, 25′, 26′ to the Internet 27. In a general configuration of FIG. 2 the central server station 20 receives and stores all the data from the connected home hubs (subsystem control units) and provides access to information about the health condition of all individuals included in the system to the authorized monitoring persons.

In this configuration of the system for determining health condition parameters of individuals remotely, the subsystem control unit 10 is connected to at least one measuring unit 16, 17 for performing continuous measurement of behavioral data of the individuals to be monitored in addition to the distinct measurements of vital signs in order to increase reliability of the measurement of the vital signs (see FIG. 1). The subsystem control unit 10 may be further configured to receive and store the results of the distinct measurements of vital signs and the continuous measurement of behavioral data and to validate the data representing the vital signs in dependence of the behavioral data. In this case each of the subsystem control unit 10 has to be programmed so that they are capable of not only collecting the measurement results but also validating them in dependence of the collected behavioral data. Validation of the data representing the vital signs means a decision whether the vital sign measuring results are acceptable and reliable for assessing the health state of the individuals to be monitored.

In an alternative embodiment, the central data server station 20 is configured to receive and store the results of the distinct measurements of vital signs and the continuous measurement of behavioral data and to validate the data representing the vital signs in dependence of the behavioral data. In this case the subsystem control units 10 need not be provided with a special validation program, and only the central data server unit 20 has to be programmed so that it is capable of collecting the measurement results, but also validate them in dependence of the collected behavioral data. In both cases the monitoring persons (health care specialist, caregiver personnel, family members, etc.) having a right to access the monitoring data, may retrieve the validated reliable monitoring data through a monitoring terminal 24, 25, 26 as shown in FIG. 2 from the central data server 20. In the example shown in FIG. 2, the monitoring terminal 24 is a terminal for the health care specialists. The monitoring terminal 25 is a terminal for the caregiver personnel and the monitoring terminal 26 is a terminal for the family members. Although there are only three monitoring terminals 24, 25, 26 shown in the drawing, a person skilled in the art will appreciate, that any other number of terminals may be selected arbitrarily according to the need of a specific application.

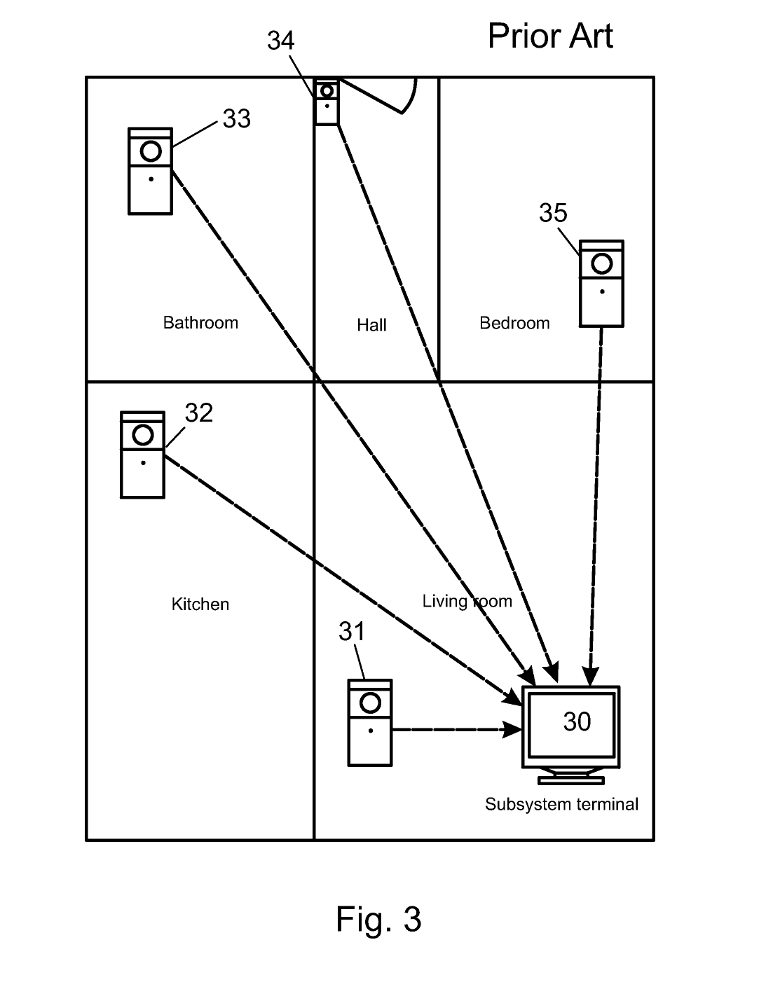

FIG. 3 shows an exemplary arrangement of the elements of a behavioral monitoring subsystem at a location of an individual to be monitored, which is may be at a distant location from the caregivers and/or the health care specialists and/or the family members. This location may be typically in the home of the individuals to be monitored, who are generally elderly people or patients released from the hospital. In this arrangement, the subsystem control unit 10 is shown as a subsystem terminal 30 with a number of fix mounted sensors 16 (contact sensors and motion sensors) that are connected to the subsystem terminal 30 through a radio or a cable communication channel. The contact sensors (not shown) are simple devices, consisting of two matching parts. A contact sensor generally can detect if these two parts are in contact or separated, and when a change happens in its state, the contact sensor sends a signal to the subsystem control unit 10. For example, if one part of this sensor is mounted to a door, and the other is to the frame of the door, the sensor can detect if the door is open or closed. Such sensors may be mounted on matching surfaces being relatively moved with respect to each other, such as a door of a room, a door of a piece of furniture or the door of equipment, such as a refrigerator. In this configuration, each room (e.g., bathroom, hall, bedroom, kitchen and living room) has its own motion detector 31, 32, 33, 34, 35, which may be wall mounted. This way, the system can provide information in which room the monitored person is located. If there are rooms, where there are well separated areas with different, meaningful purpose (for example, a living room and a kitchen in one common area), more than one motion sensor can be mounted in one room, and setup in a way to cover different areas with their field of view. These sensors 31 to 35 provide continuous measurement without any interaction of the individuals to be monitored. They are not in direct contact with the monitored person. The sensors 31, 32, 33, 34, 35 of the subsystem 21, 22, 23 are thus configured to provide continuous information regarding the location of the individual being monitored and further information on closing or opening a door of a room, a piece of furniture or equipment, that allows conclusions on the current activity of the individuals being monitored.

The body-worn activity sensors 17 of FIG. 1, that are mounted on a body part of the individuals (not shown), are also connected to the subsystem control unit 10. According to one embodiment, this is connection is though a wireless communication channel 17′. These sensors 17 provide continuous information on the activity, such as speed or acceleration of selected body parts, of the individuals to be monitored. The wireless communication link 17′ between the body-worn sensors (also called actigraph) 17 and the subsystem control unit 10 may be, for example, a communication link according to the Bluetooth, Zigbee, Wifi or other standardized specifications. In some cases, Zigbee may be preferred because of its flexibility and low power consumption.

The subsystem control unit 10, or home hub, may include a general computer provided with a suitable cable and/or radio interface units, a storage unit for storing the measured data, and input and output devices for communication with the individuals to be monitored. Such input devices may include for example, but not exclusively, a keyboard, a pointing device, such as a mouse, a touchpad, or a touch-screen, etc. The output devices may include for example, but not exclusively, a monitor, such as a flat screen monitor, a printer, an audio output device, such as a loudspeaker, etc. The interface units typically form an integral part of the subsystem control unit, but it is also possible to connect external interface units to the subsystem control unit, e.g. via a USB connector. Such a subsystem control unit is known per se and needs not to be explained further.

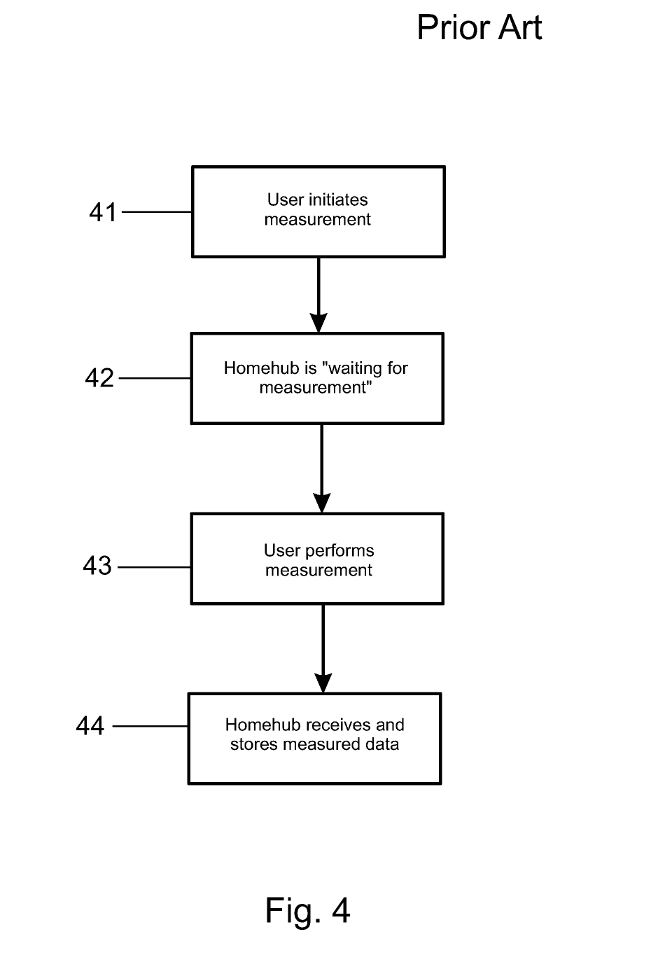

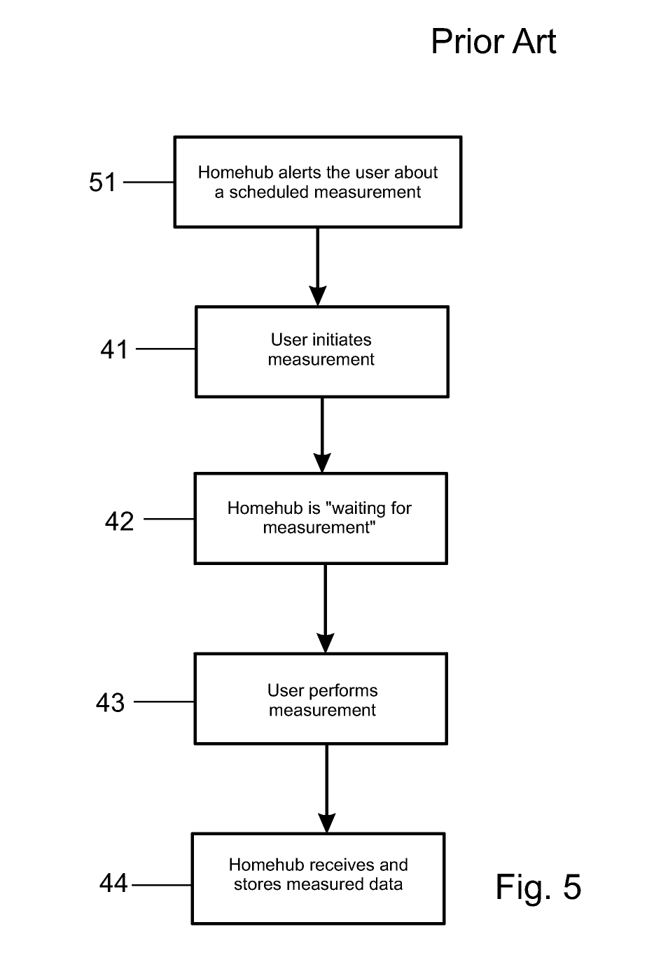

There are two scenarios, when the vital sign measurements are performed by the user, using the available measurement devices. According to a first scenario, the measurement can be initiated by the users themselves, without any instruction from the system itself According to a second scenario, if the system contains scheduling for the measurements, the initiation of a measurement can be requested by the system itself The concept described here applies to both cases, so we don't have to separately discuss them; it does not matter why a measurement has been initiated. These two scenarios are depicted in FIGS. 4 and 5 as a schematic flow diagram. FIG. 5. In the first scenario, the subsystem requires the individual to be monitored to initiate a scheduled measurement 41. This may be the case when a number of individuals have to carry out the same measurement at substantially the same time, e.g. measurement of blood pressure, temperature, weight in the morning and in the evening. In the second scenario, the home hub 10 alerts the user of a scheduled measurement 51. After the home hub 10 alerts the user of a scheduled measurement 51, all the following steps (of FIGS. 4 and 5) are identical, and therefore, identical reference signs have been used to indicate the identical steps. The individual to be monitored initiates a measurement with a vital sign measuring device not shown here connected to the home hub (e.g., by pressing a button with the label “Measure my blood pressure”) 41. In response to this, the home hub switches to a “waiting for incoming measurement” mode 42. The user performs the selected measurement with the selected device (e.g. measures his blood pressure using the blood pressure meter) 43. The home hub receives and stores the measured data (for example, the result of the vital sign measurement) 44. To this end the measurement device initiates the connection to the home hub 10, and sends the data just measured by the individual. The home hub 10 acknowledges the received measurement result and informs the user whether the measurement was successful.

Depending on the system architecture, the measurement data is either sent to the central data server's database immediately, or can be stored in the home hub 10 temporarily, and sent (e.g., in daily packages) to the central data server unit. On the basis of the collected vital sign measurement data, it is possible to provide information on the actual condition of the individuals to be monitored to health care specialists and/or care giving personnel and/or authorized family members in the form of a visual display.

An embodiment of the method further comprises performing continuous measurement of behavioral data of the individuals to be monitored in addition to the distinct measurements of vital signs, and validating the data representing the vital signs in dependence of the behavioral data in order to increase reliability of the measurement of the vital signs.

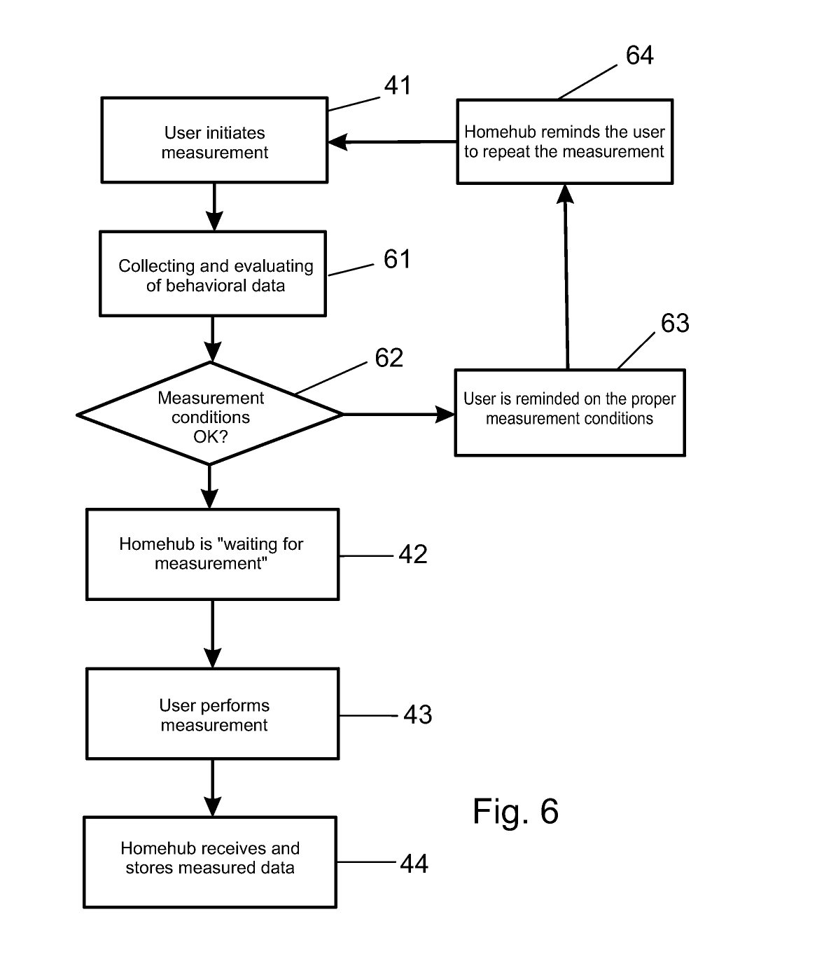

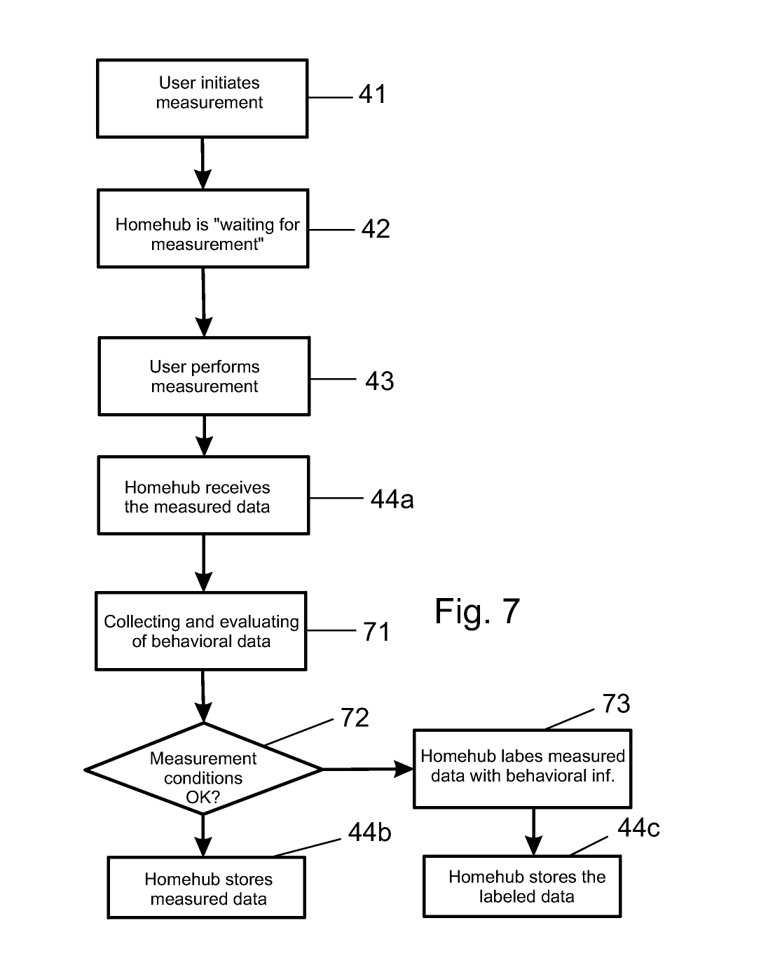

Examples of the inventive concept with respect to the above features will be explained with reference to FIGS. 6 and 7. There are two approaches to validate the measurement conditions, before and after the measurement: validating the measurement conditions before the measurement and validating the proper conditions after the measurement. Validating the measurement conditions before the measurement eliminates unnecessary measurements, and ensures more valid data measurements. It requires real-time data processing and an immediate response to the user. This means determining whether the proper conditions for the selected measurement are met or not 62 is an immediate decision, so if we continue to the user being reminded of the proper measurement conditions 63, right after the user pressed the “Measure my blood pressure” button, a message appears on the screen saying “You've had significant activity in the last few minutes, please relax and perform the measurement in 3 minutes”. Validating the proper conditions after the measurement can be performed anytime, even days later than the actual measurement, on any device. Of course, if the measurement conditions were not proper, it is not possible to repeat the measurement, and gather proper data from that moment.

The procedure to perform a measurement after validating and/or creating the proper conditions of the measurement is shown in FIG. 6. As it can be clearly seen in FIG. 6, steps 41 through 44, already discussed in connection with FIG. 4, are carried out in the same order. However, between steps 41 and 42, the steps for establishing the proper conditions for a measurement are inserted. After the user has initiated a measurement 41, the home hub 10 collects and processes the behavioral data 61 in order to make sure that the measurement conditions are appropriate. The home hub 10 then decides whether the proper conditions for the selected measurement are met or not 62. If yes, the procedure continues according to a normal measurement procedure as already explained with reference to FIG. 4. If the proper conditions for the selected measurement are not met, the home hub 10 informs the user that the conditions are not appropriate for the measurement. Furthermore, the home hub 10 informs the user of the required conditions and the actions and/or time required to achieve the required conditions 63. After a predetermined time, when the required conditions are expected to be met, the user is reminded to repeat the selected measurement 64. This leads back to initiating a measurement by the user (individual) 41.

The procedure to perform a measurement before validating or establishing the proper conditions of the measurement is shown in FIG. 7. As it can be clearly seen in FIG. 7, the steps 41 through 44 already discussed in connection with FIG. 4 are carried out in the same order. However, at step 44, the measured vital sign data are only received and not stored in order to be transferred to the central data server and for being retrieved by the monitoring persons. After step 44, the steps for examining and validating the proper conditions for a measurement are introduced. The home hub 10 collects and processes the behavioral data 71 in order to make sure that the conditions at the time of the measurement were appropriate. The home hub 10 decides whether the proper conditions for the selected measurement were met or not 72. If yes, the procedure continues according to a normal measurement procedure, and the validated measurement result is stored 44b. If, however the proper conditions for the selected measurement are not met, the home hub 10 labels the measurement data 73 and stores the measurement data with the additional data labels describing the measurement conditions (including all information useful for the person evaluating the results) 44c.

As explained above, embodiments of the present invention provide a method and a system for carrying out the method based on a known vital sign measurement method, with the addition, that behavioral data are collected from the monitored persons, which is used to make sure that the measurement has been made in the proper condition for the measurement and the person and all measurements according to the invention are validated or labeled with validation information. This has the following main advantages compared to the traditional methods. The method can enhance data quality by changing the monitored person's behavior in order to establish the proper measurement conditions. Even if, in an alternative, the measurement condition is not changed, additional behavioral information (labeled data) makes collected data more useful for the person evaluating it.

The following examples are intended to provide a better understanding of the proper measurement conditions.

### EXAMPLE 1

### Weight Measurement

Weight can change significantly depending on whether it was measured before or after eating, drinking or bathroom visits. The most appropriate moment to measure weight for a monitored person is in the morning, after the first bathroom visit, and before eating or drinking Using the mounted behavioral monitoring system, it is possible to detect whether the measurement has been taken with these conditions.

In this case, reminding the monitored person of the proper conditions means that it has been detected that no bathroom visits happened yet after waking up, and the individual is reminded to perform the measurement after visiting the bathroom (see FIG. 6).

### EXAMPLE 2

### Blood Glucose Measurement

Blood glucose levels are absolutely dependent on food intake. Using the mounted behavioral monitoring system, with sensors applied to possible food sources (e.g., fridge, closet, etc.) it is possible to detect if the monitored person potentially had food intake, which does not comply with the prescribed measurement conditions. In case of blood glucose measurement, establishing proper measurement conditions is usually not possible, so adding the food intake information to the data is performed (see FIG. 7)

### EXAMPLE 3

### Blood Pressure and ECG Measurement

Blood pressure and ECG measurement results are dependent on the amount and intensity of the activity performed before the measurement. The activity data can come from the body worn sensors, providing a precise measurement on both activity and intensity, or the mounted sensors, providing data on excessive movement in the living area. Also, if the mounted sensors show that the monitored person has just arrived home, the individual will be reminded to take a resting period before the measurement. In these cases, creating a proper measurement condition is easy. The monitored person just needs to rest for a few minutes. The home hub can even remind the monitored person whether the resting period was long enough, and the measurement can be performed (see FIG. 6).

The proposed method and system for providing remote health monitoring data makes it possible to improve reliability or to validate the result of measurements of vital signs, such as blood pressure, blood glucose, ECG, weight, etc. of elderly people or patients suffering from different diseases, carried out by the patients themselves at any location distant from medical assistance, such as in a home environment.

Due to this improvement, health care professionals and other care giving personnel can use more reliable data in assessing the current health state of an individual without personal consultation or clinical examination, which is faster, less expensive and therefore more effective.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for providing remote health monitoring data of an individual to be used in a health monitoring system, the method comprising:
measuring at least one vital sign of the individual;
receiving and storing data representing the at least one vital sign obtained from the measurement,
continuously measuring behavioral data of the individual;
validating the data representing the at least one vital sign based on the behavioral data; and
visually displaying information on a condition of the individual.

2. The method of claim 1, wherein measuring at least one vital sign of the individual is carried out by a vital sign measurement device.

3. The method of claim 1, wherein continuously measuring behavioral data of the individual comprises at least one of:
determining the motion and/or location of the individual in a selected area by fix mounted motion sensors; and
determining the motion of the individual by body-worn sensors, wherein the body-worn sensors sense the speed and/or acceleration of a selected body part of the individual.

4. The method of claim 3, further comprising comparing the measured behavioral data to predetermined behavioral data of the individual for each vital sign measurement, and, in case of divergence from the predetermined behavioral data, generating a message and communicating the message to the individual.

5. The method of claim 4, wherein the message comprises at least one of an audio and a visual message.

6. The method of claim 4, wherein the predetermined behavioral data is stored as a predetermined empirical value for all types of vital sign measurements.

7. The method of claim 4, wherein the message reminds the individual of the required measurement conditions and suggests to carry out the measurement at a predetermined time after the required measurement conditions have been established.

8. The method of claim 7, wherein the message reminds the individual of expiry of the predetermined time.

9. The method of claim 3, further comprising comparing the measured behavioral data to a predetermined behavioral data of the individual for each vital sign measurement, and, in case of divergence from the predetermined behavioral data, labeling the vital sign measurement with the measurement conditions.

10. A system for providing remote health monitoring data of an individual, the system comprising:
a plurality of subsystems at a location of the individual, the plurality of subsystems comprising a subsystem control unit and at least one vital sign measuring unit configured to measure at least one vital sign of the individual, wherein at least a portion of the at least one vital sign measuring unit is in communication with the subsystem control unit;
a central data server station in communication with the plurality of subsystems; and
a monitoring side terminal in communication with the central station configured to provide information for visual display,
wherein the subsystem control unit is connected to at least one behavioral data measuring unit configured to continuously measure behavioral data of the individual, and
wherein the subsystem control unit is configured to receive and store the vital sign measurement and the continuous measurement of the behavioral data and to validate the vital sign measurement based on the continuous measurement of the behavioral data.

11. The system of claim 10, wherein the at least one vital sign measuring unit is selected from the group consisting of devices for measuring the weight, temperature, blood pressure, blood glucose and ECG of the individual.

12. The system of claim 10, wherein the at least one behavioral data measuring unit comprises at least one of the following:
at least one fix mounted motion sensor configured to determine the motion and/or location of the individual in a selected area; and
at least one body-worn sensor configured to sense the speed and/or acceleration of a selected body part of the individual.

13. The system of claim 11, wherein at least a portion of the at least one behavioral data measuring unit is wirely connected to the subsystem control unit.

14. The system of claim 11, wherein at least a portion of the at least one behavioral data measuring unit is connected through wireless connection to the subsystem control unit.

15. A system for providing remote health monitoring data of an individual, the system comprising:
a plurality of subsystems at a location of the individual, the plurality of subsystems comprising a subsystem control unit and at least one vital sign measuring unit configured to measure at least one vital sign of the individual, wherein at least a portion of the at least one vital sign measuring unit is in communication with the subsystem control unit;
a central data server station in communication with the plurality of subsystems; and
a monitoring side terminal in communication with the central data server station configured to provide information for visual display,
wherein the subsystem control unit is connected with at least one behavioral data measuring unit configured to continuously measure behavioral data of the individual; and
wherein the central data server unit is configured to receive and store the vital sign measurement and the continuous measurement of the behavioral data and to validate the vital sign measurement based on the continuous measurement of the behavioral data.

16. The system of claim 15, wherein the at least one vital sign measuring unit is selected from the group consisting of devices for measuring the weight, temperature, blood pressure, blood glucose and ECG of the individual.

17. The system of claim 15, wherein the at least one behavioral data measuring unit comprises at least one of the following:
at least one fix mounted motion sensor configured to determine the motion and/or location of the individual in a selected area; and
at least one body-worn sensor configured to sense the speed and/or acceleration of a selected body part of the individual.
